(19) United States
(12) Patent Application Publication
(10) Pub. No.: US 2010/0260753 A1
(43) Pub. Date: Oct. 14, 2010

(54) AMINOFLAVONE (NSC 686288) AND COMBINATIONS THEREOF FOR TREATING BREAST CANCER

(76) Inventors: Angelika Burger, Detroit, MI (US); Edward Sausville, Silver Spring, MD (US); Phillip Shelton, Baltimore, MD (US)

Correspondence Address: CASTELLANO PLLC, P.O. Box 1555, Great Falls, VA, 22066, US

(21) Appl. No.: 12/739,407

(22) PCT Filed: Oct. 22, 2008
(86) PCT No.: PCT/US2008/080841

§ 371 Date: Apr. 22, 2010

Related U.S. Application Data
(60) Provisional application No. 60/981,849, filed on Oct. 23, 2007.

(51) Int. Cl. A61K 31/352; A61K 31/566; A61K 31/4196; A61K 31/506; A61K 39/395; A61K 31/517; A61P 35/00

(52) U.S. Cl. 424/133.1; 514/456; 514/171; 514/383; 514/274; 514/266.24

(57) ABSTRACT

Disclosed are methods of treating breast cancer by administering to a mammal aminoflavone, and optionally one or more additional anti-cancer agents. According to example embodiments, the methods include methods of treating cancers resistant to endocrine therapy.

### PRIOR APPLICATION

This application claims the benefit of U.S. Provisional Application No. 60/981,849, filed on Oct. 23, 2007.

### FIELD

The invention relates generally to the treatment of cancer, and in particular, breast cancer. According to example embodiments, compositions are provided that include aminoflavone (5-amino-2,3-fluorophenyl)-6,8-difluoro-7-methyl-4H-1-benzopyran-4-one, (NSC 686288; AF) for the treatment of breast cancer. Also provided are methods of treating breast cancer using aminoflavone, and optionally at least one additional anti-cancer agent.

### BACKGROUND

### Treatment Advances in Breast Cancer

Metastatic breast cancer is currently incurable, and novel strategies that might become useful treatments are needed. The past decade has witnessed the acceptance into clinical practice of Herceptin® directed against the HER2/neu oncoprotein, and aromatase antagonists. (Altundag et al. 2005 “Monoclonal antibody-based targeted therapy in breast cancer.” Curr Med Chem Anti-Cancer Agents 5: 99-106; Brodie A. 2003 “Aromatase inhibitor development and hormone therapy: a perspective.” Semin Oncol. 30 (4 Suppl 14): 12-22.) Despite these advances, cytotoxicity evoked by drugs directed at DNA or microtubules remains a cornerstone of breast cancer therapy. (Carrick et al. 2005 “Single agent versus combination chemotherapy for metastatic breast cancer.” Cochrane Database Syst Rev. epub: CD003372.) However, these cytotoxics are non-specific. Ideally, new breast cancer cytotoxics would engage some aspect of breast cancer biology to convey selective toxicity to breast cancer cells.

### Aryl Hydrocarbon Receptor.

The aryl hydrocarbon receptor (AhR) was initially defined as a receptor for environmental toxins such as dioxin. (Denison et al. 2003 “Activation of the aryl hydrocarbon receptor by structurally diverse exogenous and endogenous chemicals.” Ann. Rev. Pharmacol. Toxicol. 43: 309-34.) It belongs to the family of steroid hormone receptor-related receptors. In the cytoplasm it is complexed to the chaperone molecule heat shock protein (Hsp)90. Upon binding of a ligand it translocates to the nucleus where it complexes with ARNT (aryl hydrocarbon receptor nuclear translocator) protein to form a heterodimeric helix-loop-helix transcription factor which binds to DNA (See FIG. 1). The AhR:ARNT dimer has specificity for the xenobiotic response element (XRE), upstream of numerous genes regulating the metabolism of xenobiotics, including cytochrome P450 (CYPs P450) isoforms 1A1 and 1B1. Binding to DNA by AhR:ARNT occurs in a complex of as yet incompletely defined activating co-factor molecules. Following participation in transcriptional activation complexes, AhR is ultimately degraded in the ubiquitin-proteosome pathway. Although AhR is expressed throughout many organs, AhR signaling is cell specific and depending on transcriptional co-factors, type of ligand, accessibility of promoters, or availability of ARNT. Classical “normal organs” with prominent response to AhR activating ligands include the liver and lungs. (Spivack et al. 2003 “Phase I and II Carcinogen Metabolism Gene Expression in Human Lung Tissue and Tumors” Vol. 9: 6002-11; 2005 “Tissue distribution and function of the Aryl hydrocarbon receptor repressor (AhRR) in C57BL/6 and Aryl hydrocarbon receptor deficient mice.” Arch Toxicol epub: DOI 10.1007/s00204-005-0025-5).

### Estrogen Receptor-Aryl Hydrocarbon Receptor Cross Talk.

In breast cancer cells, it has been demonstrated that AhR ligands have the capacity to bind to estrogen receptor (ER) and potentially interfere with estrogen receptor signaling. (Pearce et al. 2004 “Interaction of the aryl hydrocarbon receptor ligand 6-methyl-1,3,8-trichlorodibenzofuran with estrogen receptor α.” Cancer Res. 64: 2889-97.) Conversely, it has long been known that estrogen can be metabolized by AhR driven genes such as CYP1B1 to yield toxic metabolites that in some cases have been proposed to act as genotoxins. (Liehr JG 2000 “Is estradiol a genotoxic mutagenic carcinogen?” Endocrine Reviews 21: 40-54.) This has led to the hypothesis that mutual modulation (“cross talk”) of AhR and estrogen receptor signaling functions may be possible. Indeed, it has been shown that certain AhR ligands can have antiproliferative effects alone or in conjunction with estrogen receptor antagonist administration with evidence of anti-tumor activity in breast cancer models. (Safe et al. 1999 “Development of selective aryl hydrocarbon receptor modulators for treatment of breast cancer.” Expert Opin Investig Drugs 8: 1385-96.) How estrogen and its antagonists will antagonize, have no effect, or amplify AhR-related signaling functions is an unresolved question.

### Benzothiazoles and Aminoflavone: AhR-Targeted Therapies.

Empirical screening in the NCI 60 cell line anticancer drug screen has revealed two types of molecules, the benzothiazoles (BZs) and aminoflavone (AF) that are noteworthy for differential cytotoxicity. “Sensitive” cell lines have total growth inhibition (TGI) between 0.1 and 1 μM, while “resistant” cell lines are refractory to ≧10 μM. Among the consistently sensitive cell lines to both compound classes were the ER(+) breast cancer cell lines MCF-7 and T47D. (Chua et al. 2000 “Role of CYP1A1 in modulation of antitumor properties of the novel agent 2-(4-amino-3-methylphenyl)benzothiazole (DF203, NSC674495).” Cancer Res. 60: 5196-203; Loaiza-Pérez et al. 2004 “Aryl hydrocarbon receptor activation of an antitumor aminoflavone: basis of selective toxicity for MCF-7 breast tumor cells.” Mol Cancer Ther. 3: 715-25). While certain other cell types in this screen did show susceptibility, e.g. renal cancer, in the breast cancer panel, optimal cytotoxicity of aminoflavone was seen in cell lines expressing estrogen receptor (ER(+)). Detailed mechanistic studies for both, benzothiazoles and aminoflavone have revealed that “sensitive” cells can activate AhR signaling, (Chua et al.; Loaiza-Pérez et al.) as might be expected from their flat planar nature. (Denison et al.) This causes expression of CYP1A1 and in certain cell lines CYP1B1. (Monks et al. 2003 “Genotoxic profiling of MCF-7 breast cancer cell line elucidates gene expression modifications underlying toxicity of the anticancer drug 2-(4-amino-3-methylphenyl)-5-fluorobenzothiazole.” Mol. Pharmacol. 63: 766-72.) Prior work had shown that CYP1A1 can metabolize benzothiazoles and aminoflavone to produce DNA-damaging metabolites. (Leong et al. 2003 “Antitumour 2-(4-aminophenyl)benzothiazoles generate DNA adducts in sensitive tumour cells in vitro and in vivo.” Br J Cancer 88: 470-7.14; Kuffel et al. 2002 “Activation of the antitumor agent aminoflavone (NSC 686288) is mediated by induction of tumor cell cytochrome P450 1A1/1A2.” Mol Pharmacol 62: 143-153.)

The precise nature of these metabolites remains to be elucidated in detail, but for aminoflavone mass spectroscopic evidence suggests the elaboration of a hydroxylamine functionality. Recent studies have confirmed that double strand DNA breaks occur and DNA-protein cross links are seen in aminoflavone-treated sensitive cells. (Meng et al. 2005 “DNA-protein cross-links and replication-dependent histone H2AX phosphorylation induced by aminoflavone (NSC 686288), a novel anticancer agent active against human breast cancer cells” Cancer Res. 65: 5337-44.) Emerging evidence suggests that sulfotransferases (i.e. SULT1A1), an important family of phase II enzymes that is responsible for estrogen sulfation in breast tumors, act on CYP1A1-derived aminoflavone metabolites to create further DNA-damaging potency, but AhR remains “upstream” of sulfotransferase action, and key to the activation of aminoflavone. (2005 “Selective activity of aminoflavone (NSC626288) a novel drug in phase I clinical trials is determined by cellular expression of sulfotransferase.” Clinical Cancer Research 11: B221; Spink et al. 2000 “SULT1A1 catalyzes 2-methoxyestradiol sulfonation in MCF-7 breast cancer cells.”Carcinogenesis 21:1947-57).

Intrinsically “resistant” cells have not been thoroughly characterized. However, in the case of the ER(−) breast cancer cell lines MDA-MB-435 and MDA-MB-231 it appears as if constitutive localization of the AhR to the nucleus is associated with lack of CYP1A1 activation and hence resistance to aminoflavone (Loaiza-Pérez et al.; 2006 “Response of breast cancer cell lines to aminoflavone (NSC 686288) is associated with histone H2AX phosphorylation and estrogen receptor expression” Proc Amer Assoc Cancer Res 47: 555).

FIG. 1 depicts the mode of action of aminoflavone. Aminoflavone binds to cytosolic AhR. Only breast cancer cells with cytosolic AhR can form AhR:AF complexes that translocate to the nucleus. Cells with constitutive nuclear AhR expression are not influenced by aminoflavone. In the nucleus, AhR:AF induces CYP1A1 leading to aminoflavone metabolism and DNA-damaging products. Aminoflavone sensitive breast cancer cells examined to this point express estrogen receptor (ER). In FIG. 1, SULT1A1 is sulfotransferase 1A1, and DSB means double strand breaks.

Aminoflavone has passed FDA review following a six month hiatus and is now entering National Cancer Institute-sponsored phase I studies in patients with advanced solid tumors, including breast cancer as a not further specified disease group.

### SUMMARY

Example embodiments are generally directed to treating cancer, such as breast cancer, using aminoflavone (NSC 686288; AF). Non-limiting example embodiments are directed to treating breast cancer by administering aminoflavone to humans having breast cancer cells that are resistant to endocrine therapy.

Additional non-limiting example embodiments are directed to treating breast cancer by administering aminoflavone and at least one additional anti-cancer agent to a human having breast cancer. According to non-limiting embodiments, the at least one additional anti-cancer agent may include tamoxifen, fulvestrant, anastrozole, exemestane, letrozole, capecitabine, bevacizumab, trastuzumab, and/or lapatinib.

Further embodiments include administering to a human having breast cancer cells resistant to endocrine therapy, aminoflavone and at least one additional anti-cancer agent.

### BRIEF DESCRIPTION OF THE FIGURES

Embodiments of the invention are herein described, by way of non-limiting example, with reference to the following accompanying Figures:

FIG. 1 depicts mode of action of aminoflavone.

FIG. 2 shows that in vitro and in vivo activity of aminoflavone in breast cancer cell lines correlates with estrogen receptor (ER) and aryl hydrocarbon receptor (AhR) status.

FIG. 2A is an in vitro MTT proliferation assay (5 days) showing aminoflavone activity in ER(+) and ER(−) breast cancer cell lines. FIG. 2B is an MTT assay (10 days) comparing the sensitivity of parental MCF-7 to aminoflavone and the tamoxifen resistant subclone MCF-7TAM1 to 4-OH tamoxifen (TAM) and aminoflavone respectively. FIG. 2C depicts in vivo antitumor activity of aminoflavone against MCF-7 xenografts. FIG. 2D depicts expression of AhR receptor in xenograft tissue of s.c. growing MCF-7 and MDA-MB-231 cell lines.

FIG. 3A shows that MCF-7Her2-18 expresses high levels of breast cancer resistance protein (BCRP). FIG. 3B depicts ER-α expression in aminoflavone sensitive and resistant breast cancer cell lines.

FIG. 4 depicts induction of DNA double strand breaks in aminoflavone sensitive breast cancer cells.

FIGS. 5A and 5B show that aminoflavone retains sensitivity in hormone-refractory breast cancer cell lines, such as MCF-7 Tam 1 and MCF-7/Her2-18.

FIGS. 6A and 6B show that tumor growth is inhibited when aminoflavone is administered where the breast cancer cell line is the hormone-refractory breast cancer cell line MCF-7 Tam 1.

FIG. 7 shows that cytoplasmic AhR confers aminoflavone sensitivity.

FIG. 8 shows that expression of ERα in MDA-MB-231 restores sensitivity to aminoflavone.

FIG. 9 shows that estrogen receptor antagonists enhance antiflavone activity.

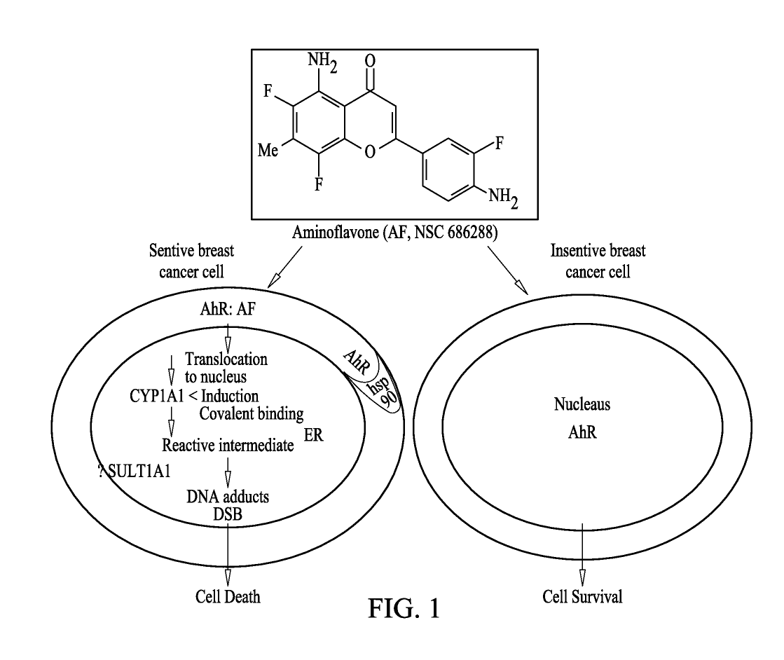

### DETAILED DESCRIPTION

The aspects, advantages and/or other features of example embodiments of the invention will become apparent in view of the following detailed description, taken in conjunction with the accompanying drawings. It should be apparent to those skilled in the art that the described embodiments of the present invention provided herein are merely exemplary and illustrative and not limiting. Numerous embodiments of modifications thereof are contemplated as falling within the scope of the present invention and equivalents thereto.

All patents and publications mentioned in this specification are indicative of the level of those skilled in the art to which the invention pertains. All patents and publications herein are incorporated by reference to the same extent as if each individual publication was specifically and individually indicated as having been incorporated by reference in its entirety.

In describing example embodiments, specific terminology is employed for the sake of clarity. However, the embodiments are not intended to be limited to this specific terminology. Unless otherwise noted, technical terms are used according to conventional usage. Definitions of common terms in molecular biology may be found, for example, in Benjamin Lewin, Genes VII, published by Oxford University Press, 2000 (ISBN 019879276X); Kendrew et al. (eds.); The Encyclopedia of Molecular Biology, published by Blackwell Publishers, 1994 (ISBN 0632021829); and Robert A. Meyers (ed.), Molecular Biology and Biotechnology: a Comprehensive Desk Reference, published by Wiley, John & Sons, Inc., 1995 (ISBN 0471186341); and other similar technical references.

As used herein, “a” or “an” may mean one or more. As used herein in the claim(s), when used in conjunction with the word “comprising”, the words “a” or “an” may mean one or more than one. As used herein “another” may mean at least a second or more. Furthermore, unless otherwise required by context, singular terms include pluralities and plural terms include the singular.

Aminoflavone (AF; NSC 686288) is an anticancer agent that has shown in vitro sensitivity toward estrogen receptor positive (ER(+)) breast cancer cell lines and in vivo activity in MCF-7 xenografts. In contrast, ER(−) breast cancer cell lines were resistant to aminoflavone. Aminoflavone is a ligand of the aryl hydrocarbon receptor (AhR). Crosstalk between AhR and estrogen receptor signaling pathways has been established. Ligand-bound AhR can mimic estrogens and redirect estrogen receptor from estrogen receptor target genes to AhR target genes, such as CYP1A1.

About 70% of breast cancer patients over-express estrogen receptor (ER(+)). About 50% of breast cancer patients that receive adjuvant endocrine therapy will develop hormone refractory metastatic disease and will require chemotherapy. All ER(+) breast cancer patients presenting with metastatic breast cancer will become hormone resistant and require chemotherapy. The present inventors discovered that aminoflavone is effective as a treatment in hormone refractory breast cancer patients.

Accordingly, present embodiments relate to the use of aminoflavone and optionally one or more additional anticancer agents in the treatment of cancer, such as breast cancer.

Non-limiting example embodiments include methods of treating breast cancer, such as by inhibiting tumor growth, that include administering aminoflavone to a human having breast cancer. According to non-limiting examples, the human may have cancer cells that are resistant to endocrine therapy, for example, breast cancer cells resistant to endocrine therapy, such as administration of tamoxifen or Herceptin®. Thus, example methods may include administering aminoflavone to a human having breast cancer cells resistant to endocrine therapy.

Non-limiting examples of breast cancer cells resistant to endocrine therapy may include for example, MCF-7 Tam1 (tamoxifen), MCF-7 Her2-18 (Herceptin®), LTLC/LTLT (letrozole). Thus, humans having one or more of these hormone-refractory breast cancer cell lines may be treated with aminoflavone and optionally at least one other anti-cancer agent in accordance with non-limiting example embodiments.

Other non-limiting example embodiments are directed to methods of treating breast cancer (e.g. in cells resistant to endocrine therapy or ER(+) cells), such as by inhibiting tumor growth, that include administering to a human having breast cancer, aminoflavone and at least one additional anti-cancer agent. Examples of additional anti-cancer agents, may include, but are not limited to, tamoxifen (e.g., Nolvadex®), fulvestrant (e.g., Faslodex®), anastrozole (e.g., Arimidex®), exemestane (e.g., Aromasin®), letrozole (e.g., Femara®), capecitabine (e.g., Xeloda®), bevacizumab (e.g., Avastin®), trastuzumab (e.g., Herceptin®), and lapatinib (e.g., Tykerb®), and/or other anti-cancer agents known to those skilled in the art.

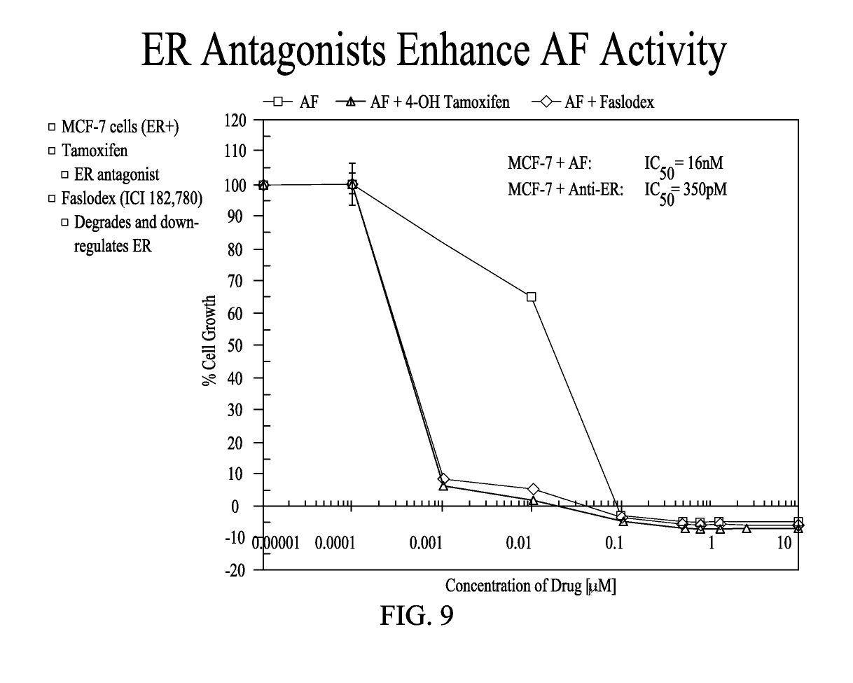

FIG. 9 demonstrates that MCF-7 (ER(+) cells) cell growth is inhibited more greatly when tamoxifen or Faslodex® is administered with aminoflavone, as compared to when aminoflavone alone is administered.

The aminoflavone may be administered to a human, as part of a composition. Thus, example methods include methods of treating cancer that include administering to a human having breast cancer, a composition that includes aminoflavone.

In embodiments where one or more additional anti-cancer agents are also administered, such additional anti-cancer agents may be administered as part of a composition that includes the aminoflavone. Alternatively, the additional anti-cancer agents may be administered separately from the aminoflavone.

Compositions that may be administered to a human in accordance herewith may include one or more “active ingredients” or “drugs.” Non-limiting example active ingredients or drugs in accordance herewith may include for example, aminoflavone, tamoxifen (e.g., Nolvadex®), fulvestrant (e.g., Faslodex®), anastrozole (e.g., Arimidex®), exemestane (e.g., Aromasin®), letrozole (e.g., Femara®), capecitabine (e.g., Xeloda®), bevacizumab (e.g., Avastin®), trastuzumab (e.g., Herceptin®), and lapatinib (e.g., Tykerb®), and other anti-cancer drugs as discussed herein. The terms “active ingredients” and “drugs” are used interchangeably herein to include any drug or other active ingredient that may be used for treating humans for a variety of different conditions including breast cancer. These terms are not meant to be limiting at all, and may include any “active ingredient” and “drug” known to those skilled in the art, which may be administered in the present methods.

The terms “active ingredients” and “drugs” are also intended to encompass analogs, prodrugs, salts, esters, polymorphs, and/or crystalline forms of active ingredients and drugs, as would be apparent to those skilled in the art.

Example compositions may optionally include one or more excipients or other ingredients as would be apparent to those skilled in the art. The term “excipient” is used herein to include pharmaceutically acceptable inert substances added to a drug formulation to give e.g., a desired consistency or form.

Aminoflavone and/or other anti-cancer agents or compositions including any of these active ingredients may be administered by methods known to those skilled in the art including, but not limited to, intraperitoneally, intravenously, orally, subcutaneously, intradermally, intramuscularly, intravascularly, endotracheally, intraosseously, intra-arterially, intravesicularly, intrapleurally, topically, intraventricularly, or through a lumbar puncture (intrathecally), or the active ingredient may be implanted in the human.

Further, non-limiting example embodiments include methods of treating cancer, such as by inhibiting tumor growth, that include administering to a human having breast cancer cells resistant to endocrine therapy, aminoflavone and at least one other anti-cancer agent. The breast cancer may include one or more hormone-refractory cell lines as discussed herein.

Further example embodiments are directed to methods of treating breast cancer that include administering to a human having breast cancer a composition that includes aminoflavone (NSC 686288) and at least one other anti-cancer agent. Alternatively, methods may include administering aminoflavone (NSC 686288) and at least one other anti-cancer agent separately.

It is contemplated that the methodology herein could be used prophylactically, prior to the onset of cancer in humans, for example in humans in a high risk group for developing breast cancer, such as those that may test positive for a breast cancer gene.

The following examples are provided to further illustrate various non-limiting embodiments and techniques. It should be understood, however, that these examples are meant to be illustrative and do not limit the scope of the claims. As would be apparent to skilled artisans, many variations and modifications are intended to be encompassed within the spirit and scope of the invention.

### EXPERIMENTAL EXAMPLES

### Example 1

### Definition of AF Sensitive and Resistant Cell Lines

The present inventors, recognizing that benzothiazoles as well as aminoflavone can activate AhR signaling, examined a set of breast cancer cell lines that was found to be sensitive or resistant to these agents. (Loaiza-Pérez et al.; Leong et al.) Isogenic subclones of MCF-7 resistant to the BZ DF203F (MCF-7DF203r), tamoxifen (MCF-7TAM1, MCF-7Her2-18) and Herceptin® (MCF-7Her2-18) were included.

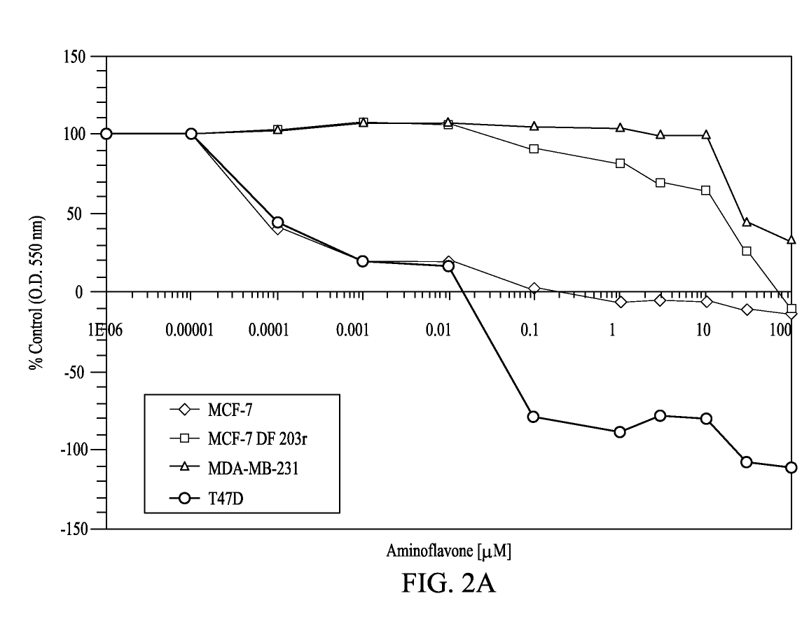

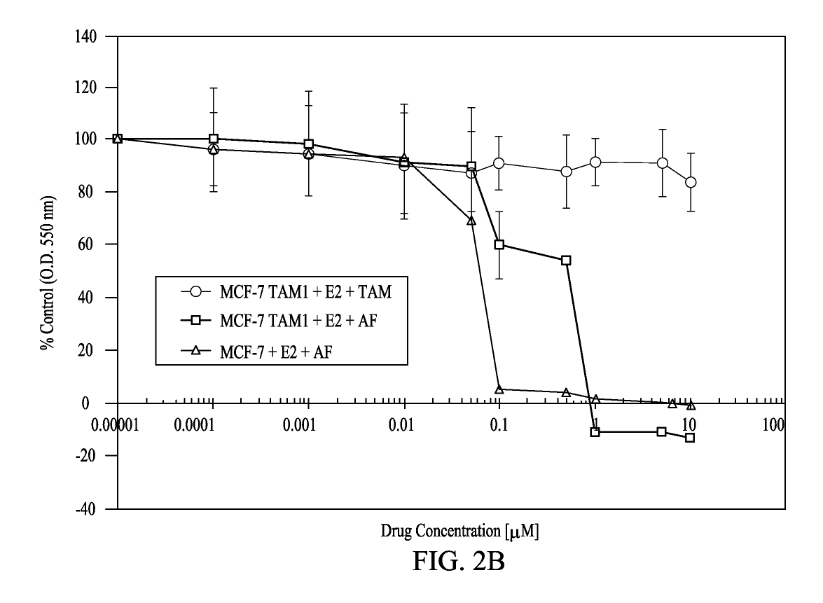

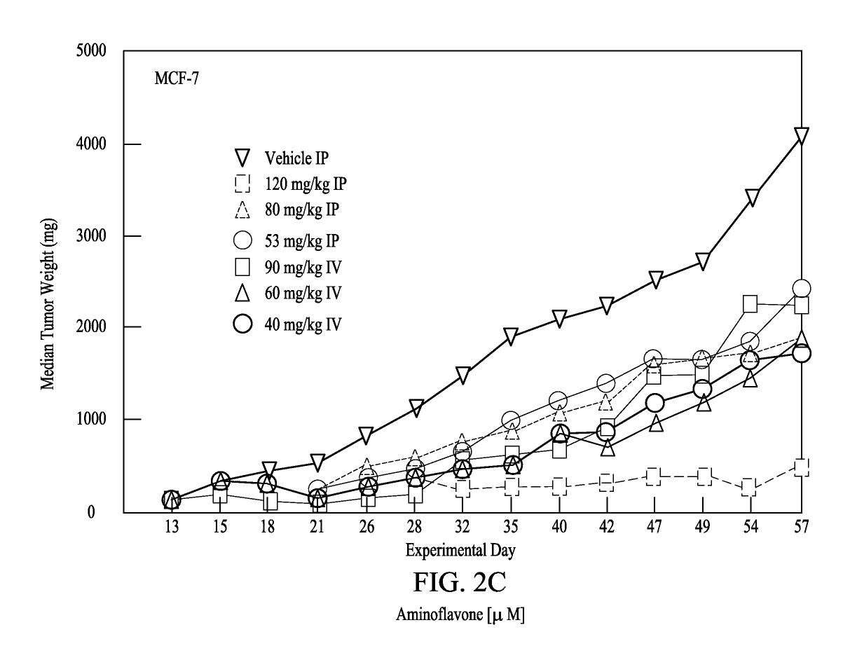

FIG. 2 demonstrates that in vitro and in vivo activity of aminoflavone in breast cancer cell lines correlates with estrogen receptor and AhR status.

FIG. 2A depicts in vitro MTT proliferation assay (5 days) showing aminoflavone activity in ER(+) and ER(−) breast cancer cell lines. FIG. 2A demonstrates that the ER(+) MCF-7 and T47D cell lines show pronounced susceptibility to aminoflavone, while the ER(−) cell line MDA-MB-231 exhibits refractoriness. The MCF-7 subclone with acquired resistance to benzothiazoles (DF203) is essentially completely cross-resistant to aminoflavone (FIG. 2A).

FIG. 2B depicts an MTT assay (10 days) comparing the sensitivity of parental MCF-7 to aminoflavone and the tamoxifen resistant subclone MCF-7TAM1 to 4-OH tamoxifen (TAM) and aminoflavone respectively. Cells were grown in medium with charcoal-stripped fetal calf serum supplemented with 10 nM E2, estrogen. MCF-7 cells that have become resistant to tamoxifen by either selection (MCF-7TAM1, FIG. 2B), or genetic manipulation (MCF-7Her2-18, Table 1), remain sensitive to aminoflavone. An understanding of how MCF-7 DF203r and MDA-MB-231 differ from MCF-7 in the dynamics and distribution of their AhR and AhR associated signaling complexes may allow the clarification of the basis for aminoflavone sensitivity and resistance.

FIG. 2C shows in vivo antitumor activity of aminoflavone against MCF-7 xenografts. Treatments were given 5 days per week for the duration of the experiment (control n=20, AF n=6). Optimal activity was seen at 120 mg/kg aminoflavone administered intraperitoneally, which led to a tumor growth inhibition of 85% of control (Loaiza-Pérez et al.) FIG. 2C data, recently reported elsewhere (Loaiza-Pérez et al.), demonstrate responsiveness of MCF-7 xenografts to aminoflavone.

FIG. 2D shows the expression of AhR receptor in xenograft tissue of s.c. growing MCF-7 and MDA-MB-231 cell lines. Rabbit immunoglobulin (IgG) was used as negative control. Heat retrieval on paraffin sections was done with citrate buffer pH 6.0. Positive reactions were developed using the EnVision Plus kit (DAKO) and diaminobenzidine as substrate (brown). Bars are 100 and 20 μm respectively. In FIG. 2D it is apparent that in the MCF-7 xenografts the untreated AhR is completely cytoplasmic in distribution, while in the insensitive MDA-MB-231 xenografts, the AhR has a prominent nuclear localization.

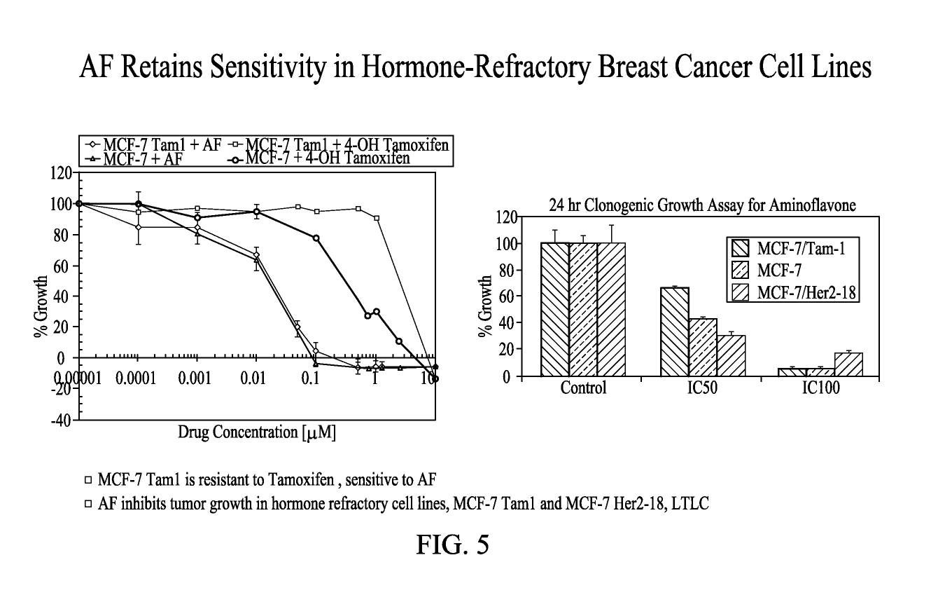

FIGS. 5A and 5B show that while MCF-7 Tam1 is resistant to tamoxifen, it is sensitive to aminoflavone. In particular, aminoflavone inhibits tumor growth in hormone refractory cell lines, MCF-7 Tam1 and MCF-7 Her2-18, LTLC.

### Example 2

### Effects of Aminoflavone in Tamoxifen Sensitive and Resistant Cells

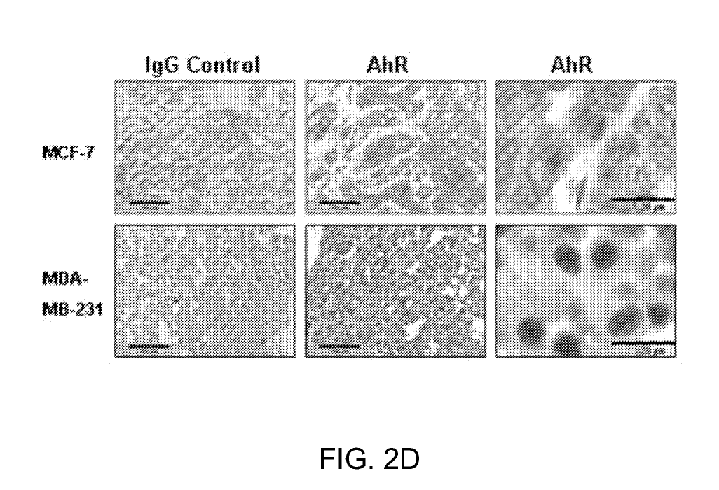

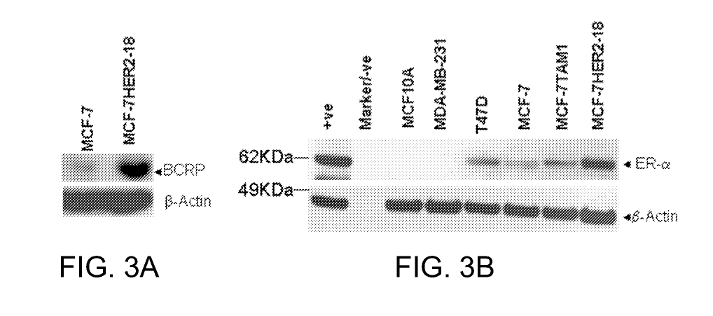

MCF-7Her2-18 is a variant of MCF-7 over-expressing the Her2/neu oncoprotein. As a result, it is resistant to Herceptin® (Shou et al. 2004 “Mechanisms of tamoxifen resistance: increased estrogen receptor-HER2/neu cross-talk in ER/HER2-positive breast cancer.” J Nat Cancer Inst 96: 926-35) and tamoxifen. (Burger et al. 2005 “Essential roles of IGFBP-3 and IGFBP-rP1 in breast cancer.” Eur. J. Cancer 41: 1515-27.) The present inventors have found that MCF-7Her2-18 has also dramatically up-regulated levels of the BCRP (breast cancer resistance protein) drug efflux pump (See FIG. 3B), rendering it resistant to anthracyclines, mitoxantrone, and the camptothecins. (1999 “A multidrug resistance transporter from human MCF-7 breast cancer cells.” Proc Natl Acad Sci USA. 95: 15665-70.) The IC₅₀s (16-20 nM) and IC₁₀₀s of aminoflavone (˜300 nM) were nearly identical in MCF-7Her2-18 and parental MCF-7 cells (See Table 1, below). In MCF-7TAM1 cells, which were serum starved and then treated for 10 days with E2 alone, 4-OH-tamoxifen plus E2, and aminoflavone plus E2, the concentration of 4-OH-tamoxifen causing growth inhibition was >10 μM. In contrast, the cell line retains noteworthy sensitivity to aminoflavone with an IC₅₀ of 550 nM (FIG. 2B).

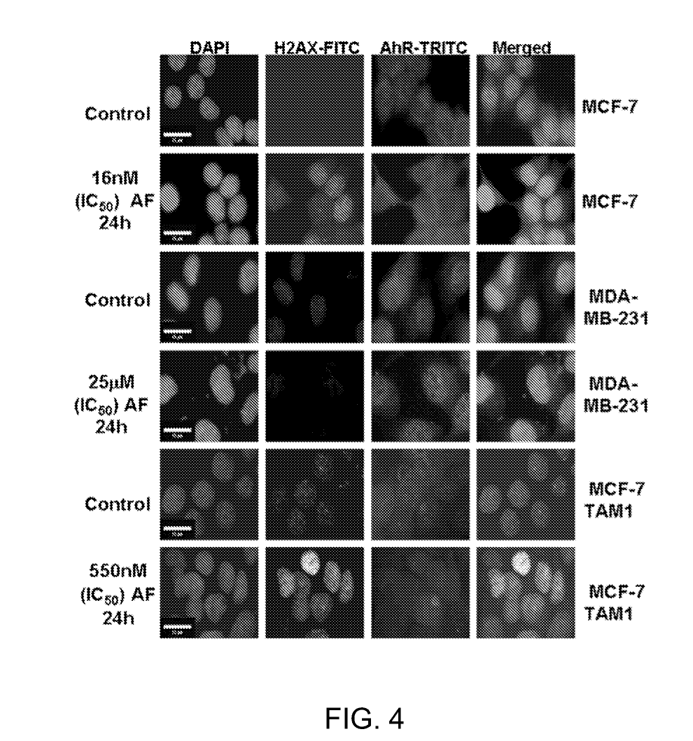

Table 1 below summarizes the results of the MTT (methyl tetrazolium) tests that were performed to assay cell growth in a panel breast cell lines and their characteristics. Both, MCF-7Her2-18 and MCF-7TAM1 express estrogen receptor. Compared to parental MCF-7, estrogen receptor levels are slightly increased (FIG. 3B) and the AhR receptor is found in the cytoplasm (Table 1, FIG. 4). The ER(−) cells MDA-MB-231 and MCF10A with nuclear AhR are several log-fold less sensitive to aminoflavone (Table 1, FIG. 4). These observations confirm that aminoflavone is valuable in patients who have lost clinical benefit from estrogen receptor antagonists.

MCF-7 and T-47D are examples of ER(+) cell lines. MCF10A, Hs578T, and MDA-MB-231 are all examples of ER(−) cell lines. MCF-7 Tam1 (tamoxifen), MCF-7 Her2-18 (Herceptin®), and LTLC/LTLT (letrozole) are all examples of hormone-refractory cell lines ER(+). Experimental results regarding several of these cell lines are depicted e.g., in FIGS. 3 and 4.

The inhibitory concentrations 50% (IC₅₀) and 100% (IC₁₀₀) listed in Table 1 represent the mean of three independent experiments. The estrogen receptor and AhR assays were performed three times.

TABLE 1

| | Cell Line | | | | | | | γ-H2AX |
|---|---|---|---|---|---|---|---|---|
| Cell Line | Characteristics | IC₅₀ | | IC₁₀₀ | | ER Status | AhR Status | Focl |
| MCF-7 | breast cancer ceil line | 16 | nM | 300 | nM | positive | cytoplasmic | yes |
| | (adenocarcinoma) | | | | | | | |
| MCF-7 HER2-18 | MCF-7 intrinsically | 20 | nM | 375 | nM | positive | cytoplasmic | yes |
| | resistant to tamoxifen | | | | | | | |
| | and Herceptin | | | | | | | |
| MCF-7 TAM1 | MCF-7 acquired | 550 | nM | 2 | μM | positive | cytoplasmic | yes |
| | resistance to tamoxifen | | | | | | | |
| T47D | breast cancer cell line | 14 | nM | 20 | nM | positive | cytoplasmic | yes |
| | (infiltrating ductal | | | | | | | |
| | carcinoma) | | | | | | | |
| MDA-MB-231 | invasive breast cancer | 25 | μM | >100 | μM | negative | nuclear | no |
| | line (adenocarcinoma) | | | | | | | |
| MCF10A | Immortal, normal | 3 | μM | 9 | μM | negative | nuclear | no |
| | breast cell tine | | | | | | | |

FIG. 3A shows that MCF-7Her2-18 expresses high levels of breast cancer resistance protein (BCRP). FIG. 3B shows estrogen receptor-α expression in aminoflavone sensitive and resistant breast cancer cell lines.

### Example 3

### Histone 2AX Phosphorylation as a Marker of Aminoflavone Effect

Previous studies have reported that the occurrence of foci of phosphorylated γ-H2AX is indicative of the cellular response to aminoflavone in MCF-7 cells (Meng et al.). γ-H2AX phosphorylation is an early indicator of DNA double strand breaks and thus, induction of DNA-damage response in cells.

FIG. 4 demonstrates the induction of γ-H2AX phosphorylation in aminoflavone responsive MCF-7 and MCF-7TAM1 cell lines at their IC₅₀s (16-550 nM), and absence of such foci in aminoflavone refractory MDA-MB-231 cells (See Table 1). Notably, the ER(−) MDA-MB-231 and MCF10A cell lines with predominately nuclear AhR are poorly inhibited by aminoflavone (IC₅₀s 3-25 μM). ER(+) cell lines with cytosolic AhR, albeit resistant to standard breast cancer therapeutics, retain the capability of DNA-damage induction and hence demonstrate exquisite sensitivity to aminoflavone (FIG. 4, Table 1).

FIG. 4 demonstrates induction of DNA double strand breaks in aminoflavone sensitive breast cancer cells. Phosphorylation of the histone variant γ-H2AX is a rapid and sensitive response to DNA double strand breaks. Aminoflavone induces γ-H2AX foci in drug sensitive MCF-7 and MCF-7TAM1 cells at IC₅₀ concentrations 24 hrs after exposure, but not in aminoflavone resistant MDA-MB-231 breast cancer cells. γ-H2AX was detected by using a FITC-labeled anti-mouse secondary antibody (green) and its nuclear expression is shown by co-localization with the nuclear stain DAPI (blue). AhR was detected with a TRITC-labeled (red) secondary antibody.

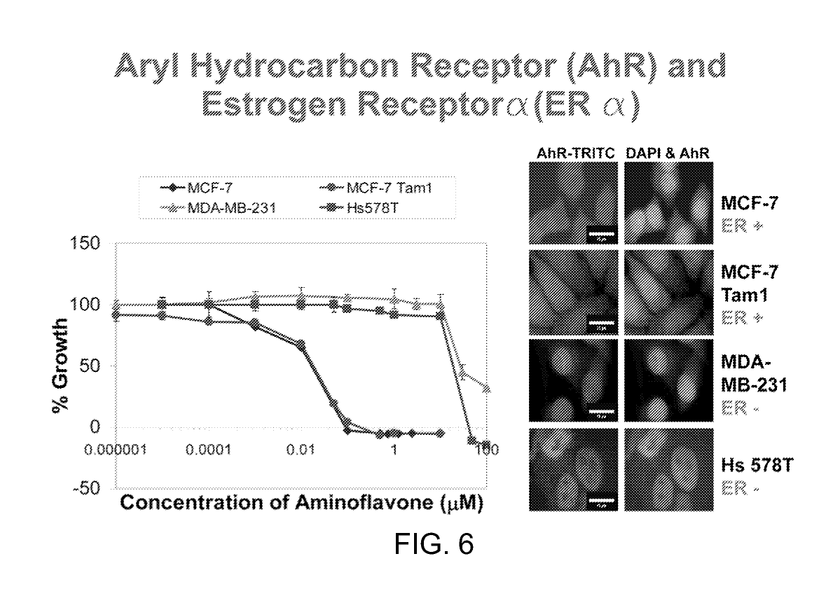

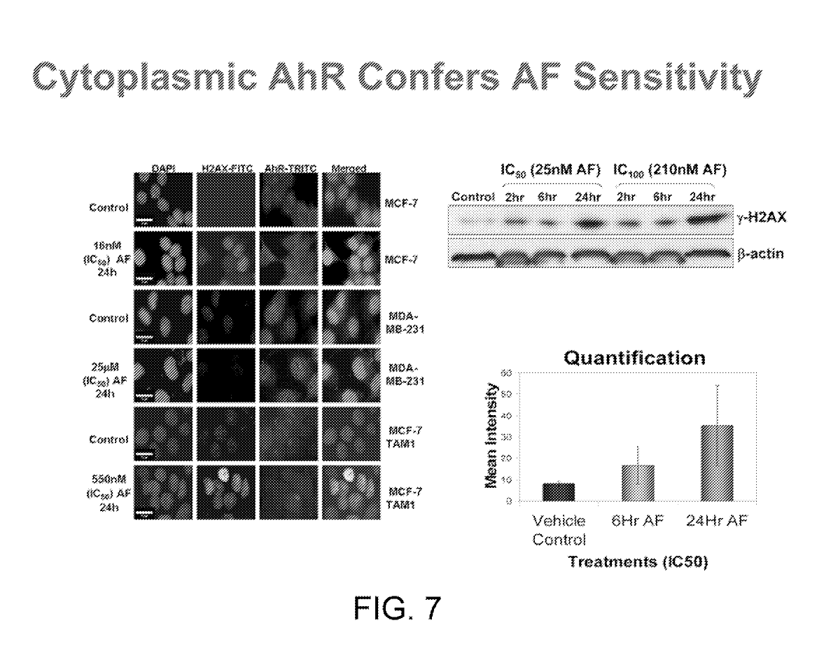

FIGS. 6A and 6B also show that MCF-7 Tam 1 cells are sensitive to aminoflavone as compared to MDA-MD-231 and Hs578T, which is aminoflavone resistant.

### Example 4

### Aminoflavone Combination with Estrogen Receptor Antagonists

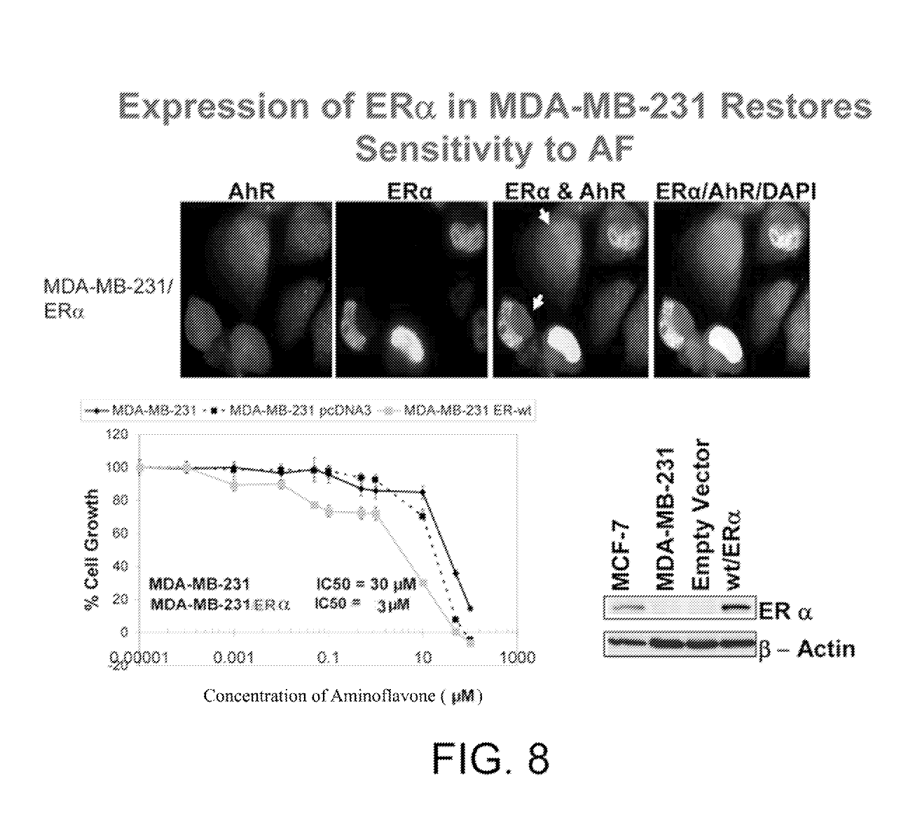

To examine the role of estrogen receptor in aminoflavone sensitivity, the inventors combined aminoflavone with a fixed concentration (100 nM) of the “pure” antiestrogen Faslodex® in MCF-7 cells. The IC₅₀ for aminoflavone plus Faslodex® was found to be 0.5 nM, unexpectedly showing a synergism between the two drugs. To further prove that ER-AhR crosstalk is correlated with aminoflavone sensitivity, MDA-MB-231 cells (ER(−)) were stably transfected with human estrogen receptor-α, rendering them ER(+). The inventors found that the ER+MDA-MB-231 cells had cytoplasmic AhR and were 5-times more sensitive to aminoflavone (IC₅₀=5 μM) compared to parental and vector transfected cells. FIG. 8 demonstrates that the expression of estrogen receptor-α in MDA-MB-231 restores sensitivity to aminoflavone.

In view of the above non-limiting example embodiments are directed to methods that include transfecting ER(−) cells with human estrogen receptor-α, rendering the cells ER(+). Such methods may further include administering aminoflavone, and optionally one or more additional anti-cancer agents, to a mammal having such cells, to treat cancer, for example, by inhibiting tumor growth. The ER(−) cells that are converted to ER(+) cells, may be those ER(−) cells disclosed herein, such as MDA-MB-435 cells or MDA-MB-231 cells, or other ER(−) cells that may be known to those skilled in the art.

Although the invention has been described in example embodiments, those skilled in the art will appreciate that various modifications may be made without departing from the spirit and scope of the invention. It is therefore to be understood that the inventions herein may be practiced other than as specifically described. Thus, the present embodiments should be considered in all respects as illustrative and not restrictive. Accordingly, it is intended that such changes and modifications fall within the scope of the present invention as defined by the claims appended hereto.

What is claimed is:

1. A method of treating breast cancer comprising administering aminoflavone to a human having breast cancer cells resistant to endocrine therapy.

2. The method of claim 1, wherein said breast cancer cells comprise cells from one or more hormone-refractory cell lines selected from the group consisting of MCF-7 Tam1, MCF-7 Her2-18, and LTLC/LTLT.

3. The method of claim 1, further comprising administering to the human at least one additional anti-cancer agent.

4. The method of claim 3, wherein said aminoflavone and the at least one additional anti-cancer agent are administered to the human as part of a composition comprising both the aminoflavone and the at least one additional anti-cancer agent.

5. The method of claim 3, wherein the aminoflavone and the at least one additional anti-cancer agent are administered to the human separately from one another.

6. The method of claim 3, wherein the at least one additional anti-cancer agent comprises at least one anti-cancer agent selected from the group consisting of tamoxifen, fulvestrant, anastrozole, exemestane, letrozole, capecitabine, bevacizumab, trastuzumab, and lapatinib.

7. The method of claim 1, wherein the aminoflavone is aminoflavone NSC 686288.

8. A method of treating breast cancer comprising administering to a human having breast cancer, aminoflavone and at least one additional anti-cancer agent.

9. The method of claim 8, wherein the aminoflavone and the at least one additional anti-cancer agent are administered to the human as part of a composition comprising both the aminoflavone and the at least one additional anti-cancer agent.

10. The method of claim 8, wherein the aminoflavone and the at least one additional anti-cancer agent are administered to the human separately from one another.

11. The method of claim 8, wherein the at least one additional anti-cancer agent comprises at least one anti-cancer agent selected from the group consisting of tamoxifen, fulvestrant, anastrozole, exemestane, letrozole, capecitabine, bevacizumab, trastuzumab, and lapatinib.

12. The method of claim 8, wherein said breast cancer is resistant to endocrine therapy.

13. The method of claim 12, wherein said breast cancer cells comprise cells from one or more hormone-refractory cell lines selected from the group consisting of MCF-7 Tam1, MCF-7 Her2-18, and LTLC/LTLT.

14. The method of claim 8, wherein the human has estrogen receptor (ER) positive breast cancer cells.

15. The method of claim 8, wherein the aminoflavone is aminoflavone NSC 686288.

16. A method of inhibiting tumor growth comprising administering to a human having breast cancer cells resistant to endocrine therapy, aminoflavone and at least one additional anti-cancer agent.

17. The method of claim 16, wherein said breast cancer cells comprise cells from one or more hormone-refractory cell lines selected from the group consisting of MCF-7 Tam1, MCF-7 Her2-18, and LTLC/LTLT.

18. The method of claim 16, wherein said aminoflavone and the at least one additional anti-cancer agent are administered to the human as part of a composition comprising both the aminoflavone and the at least one additional anti-cancer agent.

19. The method of claim 16, wherein the aminoflavone and the at least one additional anti-cancer agent are administered to the human separately from one another.

20. The method of claim 16, wherein the at least one additional anti-cancer agent comprises at least one anti-cancer agent selected from the group consisting of tamoxifen, fulvestrant, anastrozole, exemestane, letrozole, capecitabine, bevacizumab, trastuzumab, and lapatinib
